(19) United States
(12) Patent Application Publication
(10) Pub. No.: US 2014/0371668 A1
(43) Pub. Date: Dec. 18, 2014

(54) ELECTRICAL APPARATUS

(71) Applicant: SMITHS MEDICAL INTERNATIONAL LIMITED, Kent (GB)

(72) Inventors: Michael Welsch, St. Paul, MN (US); Larry R. Zalesky, Milaca, MN (US)

(73) Assignee: SMITHS MEDICAL INTERNATIONAL LIMITED, Kent (GB)

(21) Appl. No.: 14/368,533

(22) PCT Filed: Dec. 12, 2012
(86) PCT No.: PCT/GB2012/000898

§ 371 Date: Jun. 25, 2014

(30) Foreign Application Priority Data
Jan. 7, 2012 (GB) 1200297.8

(51) Int. Cl. A61M 5/168; G06F 13/10; A61M 5/145

(52) U.S. Cl. CPC A61M 5/16831; A61M 5/1452; G06F 13/10
USPC 604/67; 604/151; 710/62

(57) ABSTRACT

A medical syringe pump has a first processor (21) responsible for controlling infusion and a second processor (22) that functions as a watchdog to monitor operation of the first processor. A data communication connector (27) is located in the pump battery compartment (25) so that the batteries (24) have to be removed before external access can be made using an external connector (4). Removing the batteries (24) disables the infusion function of the first processor and ensures that the pump is prevented from infusing medication. When the external connector (4) is connected the first processor (21) can reprogram the second processor (22) or information about historical operation of the pump can be downloaded.

This invention relates to electrical apparatus of the kind including processing means and a first electrical connector for establishing data transfer communication between the processing means and an external device via a second, mating connector.

The invention is more particularly concerned with battery-powered apparatus such as medical infusion pumps.

Most modern medical infusion pumps include an arrangement for communication to or from the pump, such as for use in programming the pump or downloading pump history or other information to an external computer. The usual way in which this communication is established is by means of a wired connection, such as to an electrical connector or port on the pump housing, and via a USB or Ethernet communications protocol. There can be problems in making such a wired communication to an infusion pump. One problem arises from the possibility that, if such a communication were made while the pump was in operation supplying medication to a patient it could adversely affect operation of the pump. There could also be the risk of shock or leakage currents flowing to the pump from the external computer. It is important, therefore, to ensure that the pump is disabled from critical functions during external communication. Conventional arrangements for disabling pumps can be effective but can be relatively complex and expensive, making them unsuitable for use in low-cost pumps. There are also other electrical apparatus, apart from medical infusion pumps, where similar considerations apply. Another problem arises with apparatus including one or more processors arranged to operate in different modes, in that it can be important to ensure that the or each processor is in the appropriate mode during particular operations.

Electrical devices are known that can be operated either by internal battery power or from an external power supply via a cable. In order to prevent excessive power being delivered it is known, such as from CN2812312, to locate the connector for the external power cable in the battery compartment so that connection can only be made after the batteries have been removed.

It is an object of the present invention to provide alternative electrical apparatus.

According to one aspect of the present invention there is provided electrical apparatus of the above-specified kind, characterised in that the first connector is located on the apparatus in such a manner that the second connector cannot be connected with it while a removable component of the apparatus remains assembled on the apparatus and such that removal of the component to provide access to the first connector disables at least a function of the apparatus before and after connection of the second connector.

According to another aspect of the present invention there is provided an arrangement including electrical apparatus including processing means, a first electrical connector, an external device, and an electrical cable extending from the external device and terminated by a second electrical connector mateable with the first electrical connector to establish data transfer between the external device and The processing means, characterised in that the first connector is located on the apparatus in such a manner that the second connector cannot be connected with it while a removable component of the apparatus remains assembled on the apparatus and such that removal of the component to provide access to the first connector disables at least a function of the apparatus both before and after connection of the second connector.

The removable component may be one or more batteries in a battery compartment. The apparatus may be a medical infusion pump, the processing means being arranged to control infusion of medication from the pump.

According to a further aspect of the present invention there is provided a method of communicating data with electrical apparatus including the steps of removing a component of the apparatus so as to disable an operation of the apparatus and to enable access to a first electrical connector on the apparatus that is only accessible after removal of the component, and connecting a second electrical connector electrically connected with an external device with the first electrical connector such that the external device is put in data communication with the processing means in the electrical apparatus.

According to yet another aspect of the present invention there is provided a medical infusion pump of the kind including a housing, a medication reservoir, means for expelling medication from the reservoir to a patient, processing means operable to control the means for expelling medication, and an electrical connector on the housing, characterised in that the connector is in a location where it is inaccessible during infusion but can be accessed after removal of a component of the pump to enable data communication with the processing means in the pump, and that the pump is arranged to prevent infusion operation by the processing means after removal of the component even after connection is made to the connector.

According to a fifth aspect of the present invention there is provided a medical infusion pump of the kind including a housing, a medication reservoir, means for expelling medication from the reservoir to a patient, processing means operable to control the means for expelling medication, and an electrical connector on the housing, characterised in that the pump is operable in an infusion mode or a communications mode, that the connector is in a location where it is inaccessible during infusion but can be accessed after removal of a component of the pump, and that removal of the component disables the infusion mode of the pump and enables data communication with the pump in its communication mode via the connector.

The medication reservoir may be a syringe and the means for expelling medication may include a motor operable to rotate a lead screw that is engaged by a syringe pusher so that rotation of the motor effects movement of the pusher to expel medication from the syringe. The processing means may include two processors, one processor being arranged to control medical, infusion functions of the pump, that the other processor being arranged to monitor functioning of the first processor, removal of the component disabling the medical, infusion functions of the one processor and enabling it to program the other processor. The processing means may include two processors, one processor being arranged to control medical, infusion functions of the pump, that the other processor being arranged to monitor functioning of the first processor, removal of the component disabling the medical, infusion functions of the one processor and enabling downloading of information about the history of operation of the pump.

A medical infusion, syringe pump arrangement and its method of use, in accordance with the present invention, will now be described, by way of example, with reference to the accompanying drawings, in which:

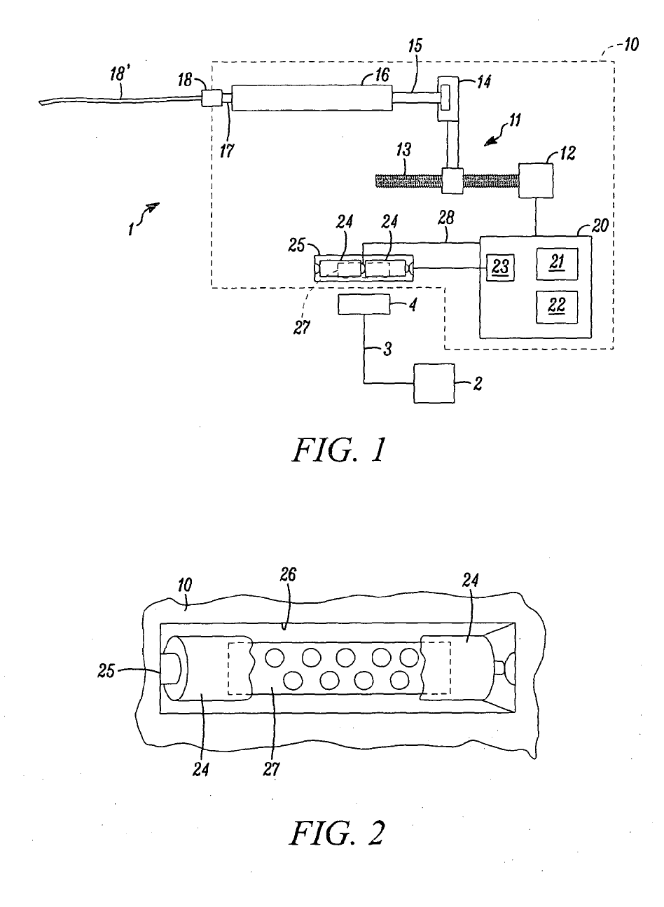

FIG. 1 is a schematic view of the pump arrangement; and

FIG. 2 is a perspective view of the battery compartment of the pump.

The arrangement shown in FIG. 1 comprises a syringe pump 1, an external device in the form of a computer 2 and an electrical data communications cable 3 extending from the computer and terminated by a connector 4. The arrangement is such that the connector 4 on the cable 3 cannot be connected to the pump 1 while the pump is in its medical/infusing operational mode.

The pump 1 is conventional in most respects, including a housing 10 containing a drive assembly 11 including a motor 12 coupled to drive a lead screw 13, which is engaged by a syringe head pusher 14. The pusher 14 contacts the head of a plunger 15 of a medication reservoir in the form of a syringe 16 gripped securely on the front face of the pump housing 10. The nose 17 of the syringe 16 is fitted to a connector 18 at one end of an infusion catheter 18′, which extends to a patient in the usual way. The motor 12 is driven by signals from a control unit 20 to provide the desired rate of expulsion of medication from the syringe 16 to the patient. As so far described, the pump is conventional.

The control unit 20 includes two processors 21 and 22 and a power supply 23. The power supply 23 is connected to receive an input voltage from batteries 24 in a battery compartment 25 on the rear face of the housing 10. The battery compartment 25 takes the form of an elongate recess 26 (FIG. 2) containing typically two or more AA batteries in a series arrangement. The compartment 25 is normally closed by a lid (not shown), with a gasket or other seal between the housing and the lid to prevent ingress of moisture and particles to the compartment. At the bottom of the recess 26 there is mounted an electrical data connector 27 of the same type and mateable with the connector 4 at the end of the cable 3, such as a USB or Ethernet connector. The connector 27 is located beneath the batteries 24 so that they must be removed in order to access the connector, that is, in order to mate the cable connector 4 with it. It will be appreciated that the connector in the battery compartment need not be located directly below the batteries but could be located at any position where it was not possible to make full mating interconnection with a mating connector while the batteries were present. The connector 27 is connected via a cable 28 to the processors 21 and 22 in the control unit 20.

It will be appreciated that the pump could also include conventional control and safety features not mentioned, such as for detecting occlusions, absence of a syringe, end of travel of the syringe plunger, bubbles in the infusion path and so on.

The control unit 20 is operable in one of two distinct alternative modes, namely a first, “medical” or “infusion” mode and a second, “communications” or “programming” mode. In the first, infusion mode the pump 1 is operable to infuse medication to the patient at a controlled rate according to the settings of the pump. This infusion mode can only be entered if the batteries 24 are present correctly in the battery compartment 25. When the batteries 24 are not present, the pump automatically reverts to the alternative communications mode even after the external connector 4 has been connected to the connector 27 on the pump. Thus, in order to connect the computer 2 to the pump 1, the batteries 24 must be removed to enable the two connectors 4 and 27 to be mated, causing the pump automatically to revert to the second mode. The first or primary processor 21 in the pump is arranged to carry out the medical, infusion functions of the pump. The second or secondary processor 22 functions as a monitor or watchdog unit to check operation of the first processor 21 and generally to provide safety checks on operation of the pump. When the “medical/infusion” mode is disabled, by removing the batteries 24 it is possible for the primary processor 21 to reprogram the secondary processor 22. In this mode the pump derives its power from the external or host device 2 but the infusion mode remains disabled after connection of the connector 27. If this reprogramming were, instead, possible when the primary processor 21 was controlling infusion there would be the risk that the secondary processor 22 would not be able to identify a breach of safety or correct operation. By ensuring that the medical/infusion operation of the primary processor 21 is disabled automatically when an external device is connected it ensures that the safe operation of the pump is not compromised.

The present invention enables the secondary, safety processor to be reprogrammed safely in the field without the need to return the pump to the manufacturer. This saves cost for both the manufacturer and the user.

Connecting the second connector 27 to the first 4 also enables pump history data to be downloaded from the pump 1 to the external computer 2 using conventional off-the-shelf software stacks. It is well known that off-the-shelf software used in communications is complex and has a high risk of errors. By preventing communications during normal infusion operation of the pump the risk to correct operation of the pump during infusion is reduced or eliminated. Communications operation can place a large load on data processing capabilities even when behaving properly. Because there is no requirement, in the present invention, for the processor to be capable of handling both the communications and infusion control at the same time there is no need to provide additional processing to be able to handle both functions simultaneously, thereby reducing costs. Power to enable the control unit 20 to connect the primary processor 21 with the programming port of the secondary processor 22 is derived from a power line in the connectors 4 and 27, such as the 5V contact in a USB connector. It will be appreciated that alternative data communication arrangements could be used, such as ethernet, Mac Thunderbolt, Firewire or the like.

In addition to enabling safe reprogramming of the secondary processor 22, the present invention ensures that the pump is not in infusion operation when connected with the cable 3, such as when downloading the history of operation of the pump to the external computer 2. This ensures that there is no risk of anomalous signals on the cable 3 adversely affecting the pump 1 during infusion. This avoids the need to have to isolate the patient electrically from the pump communications interface, which enables the pump arrangement to be more compact and less obtrusive as well as enabling costs to be minimised.

As previously mentioned, concealing the pump connector, such as within the battery compartment, enables it to be protected from fluid or particle ingress. However, it also protects the connector from the effects of electrostatic discharge externally of the pump that might be conducted to a more exposed connector, thereby reducing the risk of damage to the pump electronics and avoiding the need for expensive and bulky filtering.

Locating the connector in the battery compartment has various advantages but it would also be possible to locate the connector in a different location where access to the connector is prevented by the presence of some other removable component of the pump, providing the pump could not be operated to infuse without that component being present. For example, the connector could be located beneath the syringe, or, in the case of other infusion pumps, beneath some other medication reservoir or other disposable so that this had to be removed before making electrical connection.

The invention is not confined to medical infusion pumps but could be used in other medical or non-medical apparatus. For example, the invention could be used in a medical therapy or treatment device. The present invention could be used in apparatus having a removable, plug-in battery unit where the batteries are contained within a housing that can be removed from the apparatus, such as to enable recharging. In such an arrangement, the first connector would be located on a part of the apparatus housing that was normally concealed by the presence of the battery unit, such that the battery unit has to be removed before the first connector can be accessed and connected with the second connector. Although the invention is primarily intended for use with battery-powered apparatus it could also be used in mains-powered apparatus by locating the electrical communications connector adjacent a power input socket such that the power cable has to be disconnected from the apparatus in order to access the first connector.

1-10. (canceled)

11. Electrical apparatus including processing means and a first electrical connector for establishing data transfer communication between the processing means and an external device via a second, mating connector, characterized in that the first connector is located on the apparatus in such a manner that the second connector cannot be connected with it while a removable component of the apparatus remains assembled on the apparatus and such that removal of the component to provide access to the first connector disables at least a function of the apparatus before and after connection of the second connector.

12. An arrangement including electrical apparatus including processing means, a first electrical connector, an external device, and an electrical cable extending from the external device and terminated by a second electrical connector mateable with the first electrical connector to establish data transfer between the external device and the processing means, characterized in that the first connector is located on the apparatus in such a manner that the second connector cannot be connected with it while a removable component of the apparatus remains assembled on the apparatus and such that removal of the component to provide access to the first connector disables at least a function of the apparatus both before and after connection of the second connector.

13. Electrical apparatus according to claim 11, characterized in that the removable component is one or more batteries in a battery compartment.

14. Electrical apparatus according to claim 11, characterized in that the apparatus is a medical infusion pump and that the processing means is arranged to control infusion of medication from the pump.

15. A method of communicating data with electrical apparatus including the steps of removing a component of the apparatus so as to disable an operation of the apparatus and to enable access to a first electrical connector on the apparatus that is only accessible after removal of the component, and connecting a second electrical connector electrically connected with an external device with the first electrical connector such that the external device is put in data communication with the processing means in the electrical apparatus.

16. A medical infusion pump of the kind including a housing, a medication reservoir, means for expelling medication from the reservoir to a patient, processing means operable to control the means for expelling medication, and an electrical connector on the housing, characterized in that the connector is in a location where it is inaccessible during infusion but can be accessed after removal of a component of the pump to enable data communication with the processing means in the pump, and that the pump is arranged to prevent infusion operation by the processing means after removal of the component even after connection is made to the connector.

17. A medical infusion pump of the kind including a housing, a medication reservoir, means for expelling medication from the reservoir to a patient, processing means operable to control the means for expelling medication, and an electrical connector on the housing, characterized in that the pump is operable in an infusion mode or a communications mode, that the connector is in a location where it is inaccessible during infusion but can be accessed after removal of a component of the pump, and that removal of the component disables the infusion mode of the pump and enables data communication with the pump in its communication mode via the connector.

18. A pump according to claim 16, characterized in that the medication reservoir is a syringe and that the means for expelling medication includes a motor operable to rotate a lead screw that is engaged by a syringe pusher so that rotation of the motor effects movement of the pusher to expel medication from the syringe.

19. A pump according to claim 16, characterized in that the processing means includes two processors, that one processor is arranged to control medical, infusion functions of the pump, that the other processor is arranged to monitor functioning of the first processor, and that removal of the component disables the medical, infusion functions of the one processor and enables it to program the other processor.

20. A pump according to claim 16, characterized in that the processing means includes two processors, that one processor is arranged to control medical, infusion functions of the pump, that the other processor is arranged to monitor functioning of the first processor, and that removal of the component disables the medical, infusion functions of the one processor and enables downloading of information about the history of operation of the pump.

21. A medical infusion pump of the kind including a housing, a medication reservoir, a drive for expelling medication from the reservoir to a patient, a processor operable to control the drive for expelling medication, and an electrical connector on the housing, characterized in that the connector is located where it is inaccessible during infusion but can be accessed after removal of a removable medication reservoir such that the medication reservoir has to be removed before connection can be made to the connector.

22. A medical infusion pump of the kind including a housing, a medication reservoir, a drive for expelling medication from the reservoir to a patient, a processor operable to control the drive for expelling medication, and an electrical connector on the housing, characterized in that the connector is in a location where it is inaccessible during infusion but can be accessed after removal of a component of the pump to enable connection of an external cable, and that the pump can only infuse medication to a patient after disconnection of the cable and replacement of the component, thereby preventing any anomalous signals on the cable being supplied to the pump during infusion.
